(19) United States
(12) Patent Application Publication
(10) Pub. No.: US 2025/0288733 A1
(43) Pub. Date: Sep. 18, 2025

(54) SYRINGE SYSTEM AND METHOD OF USING SYRINGE SYSTEM

(71) Applicant: KYOCERA CORPORATION, Kyoto-shi, Kyoto (JP)

(72) Inventor: Yutaka MORI, Kyoto-shi, Kyoto (JP)

(21) Appl. No.: 18/872,659

(22) PCT Filed: Aug. 3, 2023
(86) PCT No.: PCT/JP2023/028381

§ 371 Date: Dec. 6, 2024

(30) Foreign Application Priority Data
Aug. 25, 2022 (JP) 2022-134121

(51) Int. Cl. A61M 1/02

(52) U.S. Cl. CPC A61M 1/029

(57) ABSTRACT

To provide a syringe system with improved safety. The syringe system includes a first syringe, a first needle guide, and a first holder. The first syringe includes a first needle. The first needle guide has a first hole in which the first needle is positioned and which is shorter than a length of the first needle in a longitudinal direction. The first holder fixes the first syringe and the first needle guide at a specific interval. The specific interval is greater than a difference between a length of the first needle and a length of the first hole.

### TECHNICAL FIELD

The present disclosure relates to a syringe system and a method of using a syringe system.

### BACKGROUND OF INVENTION

Patent Document 1 discloses a device for fractionating blood (platelet rich plasma collection device).

### CITATION LIST

### Patent Literature

Patent Document 1: JP 2012-161437 A

### SUMMARY

A syringe system according to an aspect of the present disclosure includes a first syringe, a first needle guide, and a first holder. The first syringe includes a first needle. The first needle guide includes a first hole in which the first needle is positioned and which is shorter than a length of the first needle in a longitudinal direction. The first holder is configured to fix the first syringe and the first needle guide at a specific interval. The specific interval is greater than a difference between the length of the first needle and a length of the first hole.

A method of using a syringe system according to an aspect of the present disclosure includes inserting a first needle guide into a second syringe barrel in a state in which a first holder fixes a first syringe and the first needle guide at a specific interval, releasing the fixing by the first holder from the first syringe and the first needle guide in a state in which the first needle guide is positioned inside the second syringe barrel, and setting a distance between the first syringe and the first needle guide to be shorter than the specific interval in a state in which the fixing by the first holder is released from the first syringe and the first needle guide. The specific interval is greater than a difference between a length of a first hole of the first needle guide and a length of a first needle of the first syringe, and a length of the first needle in a longitudinal direction is longer than a length of the first hole in the longitudinal direction.

### BRIEF DESCRIPTION OF THE DRAWINGS

FIG. 1 is an overall view of a syringe system 100.

FIG. 2 is a cross-sectional view of the syringe system 100 taken along line A-A illustrated in FIG. 1.

FIG. 3 is a view illustrating a state in which a first syringe 10 and a first needle guide 20 are moved with respect to the syringe system 100 from FIG. 1 in a direction perpendicular to a longitudinal direction.

FIG. 4 is a front view of a first holder 30 in three directions.

FIG. 5 is an overall view of the syringe system 100 including a second syringe 40.

FIG. 6 is a cross-sectional view of the syringe system 100 including the second syringe 40 taken along line A-A illustrated in FIG. 5.

FIG. 7 is a view illustrating an example of a flow of processing of preparing PRP from blood by using the syringe system 100.

FIG. 8 is an overall view of a syringe system 200.

FIG. 9 is a cross-sectional view of the syringe system 200 taken along line A-A illustrated in FIG. 8.

FIG. 10 is a view illustrating an example of a flow of processing of preparing PRP from blood by using the syringe system 200.

FIG. 11 is an overall view of a syringe system 300.

FIG. 12 is a cross-sectional view of the syringe system 300 taken along line A-A illustrated in FIG. 11.

FIG. 13 is an overall view of a syringe system 400.

FIG. 14 is a cross-sectional view of the syringe system 400 taken along line A-A illustrated in

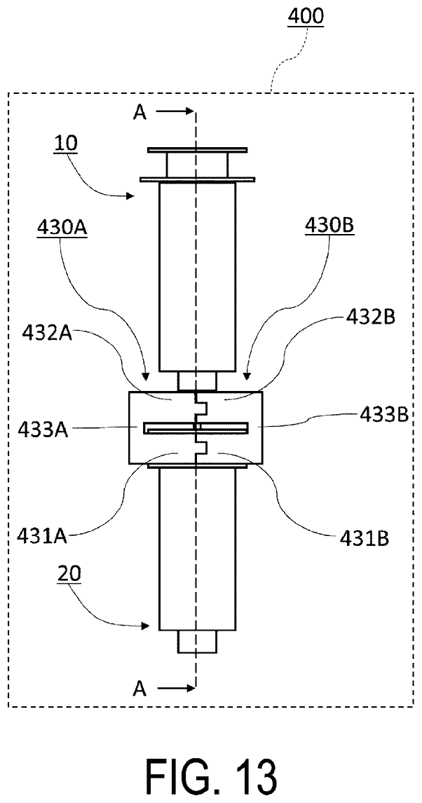

FIG. 13.

FIG. 15 is an overall view of a syringe system 500.

FIG. 16 is a cross-sectional view of the syringe system 500 taken along line A-A illustrated in FIG. 15.

FIG. 17 is a view illustrating a configuration example of a syringe system 600.

FIG. 18 is a view illustrating an example of a latter half of a flow of processing of preparing PRP from blood by using the syringe system 600.

### DESCRIPTION OF EMBODIMENTS

Hereinafter, embodiments of the present disclosure will be described with reference to the drawings.

### First Embodiment

Below, a syringe system 100 according to a first embodiment will be described with appropriate reference to the drawings. The syringe system 100 separates a specific component from a plurality of components having different specific gravities in a liquid sample. In the following description, as an example, the syringe system 100 separates platelet rich plasma (hereinafter, referred to as PRP) from blood as a liquid sample by centrifugation. However, the configuration is not limited to this, and the syringe system 100 can also be used to prepare a sample using a plurality of syringes. For example, an operator can extract a specific component from an aqueous solution sample by using an organic solvent and then separate only a specific phase using the syringe system 100.

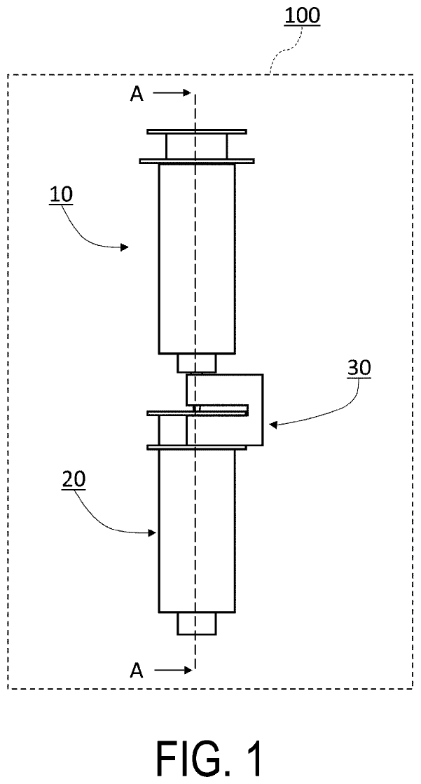

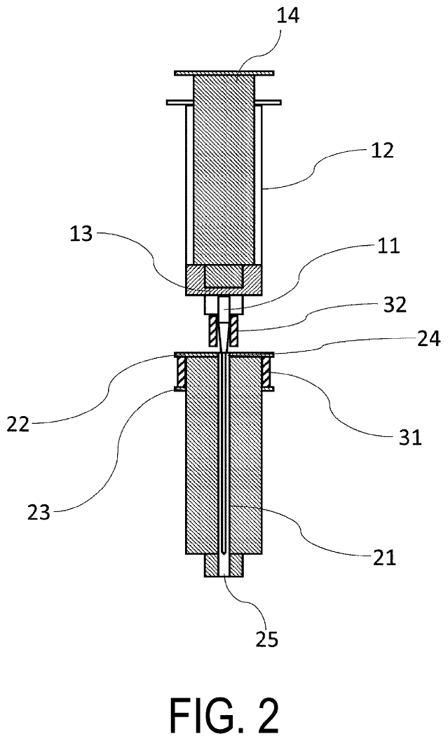

A configuration of the syringe system 100 will be described with reference to FIGS. 1 and 2. FIG. 1 is an overall view of the syringe system 100. FIG. 2 illustrates an internal structure of the syringe system 100. By using the syringe system 100, an operator can fractionate a liquid by accommodating the liquid accommodated in another syringe or the like into the syringe system 100. For example, only a specific layer of centrifuged liquid accommodated in another syringe can be accommodated in the syringe system 100. Configurations of the syringe system 100 for which plastic materials are used can be formed by injection molding, extrusion molding, or compression molding.

The syringe system 100 includes a first syringe 10, a first needle guide 20, and a first holder 30. The first syringe 10 includes a first needle 11. The first needle guide 20 has a first hole 21 in which the first needle is positioned and which is shorter than a length of the first needle in a longitudinal direction. The first holder 30 fixes the first syringe 10 and the first needle guide 20 at a specific interval L. The specific interval L is greater than a difference between a length L1 of the first needle 11 and a length L2 of the first hole 21.

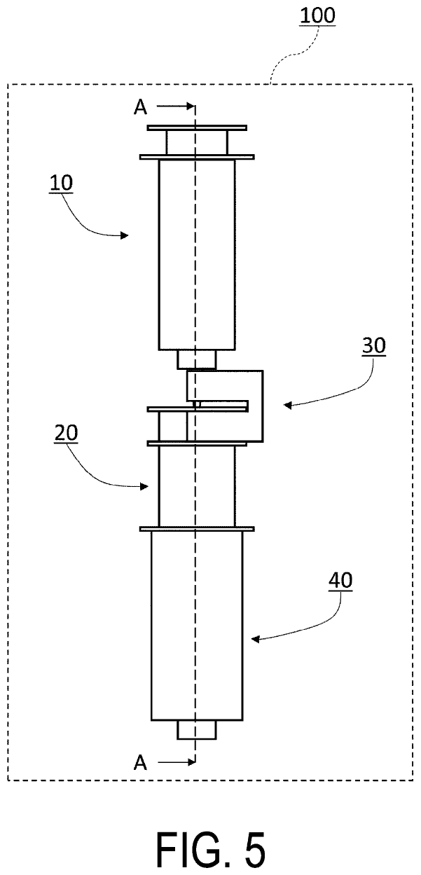

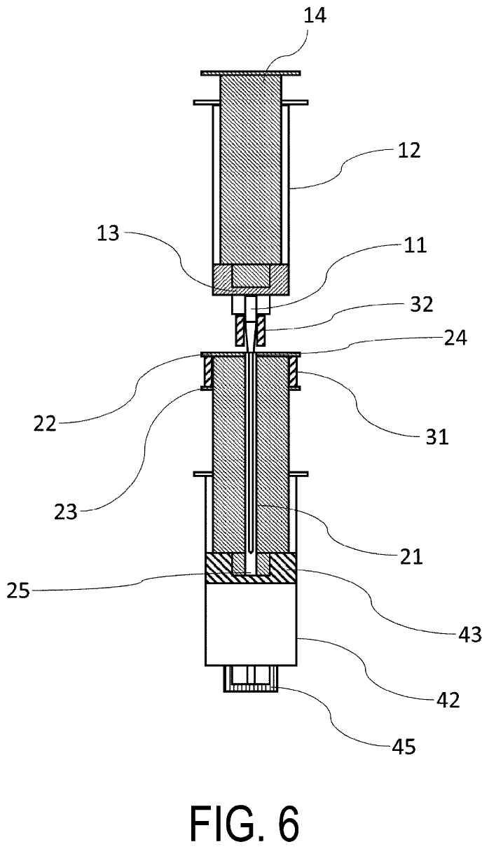

As illustrated in FIGS. 5 and 6, the syringe system 100 may further include a second syringe 40. The second syringe 40 includes a second needle 41 and a second seal 45.

The first syringe 10 can store a liquid. The first syringe 10 can store a predetermined component among a plurality of components fractionated in the second syringe 40. The first syringe 10 has the first needle 11 extending in the longitudinal direction. The longitudinal direction is approximately a height direction of a cylindrical shape of the first syringe 10, and corresponds to a direction in which the line A-A in FIG. 1 extends. The first syringe 10 may include a first syringe barrel 12 connected to the first needle 11, a first gasket 13 positionable within the first syringe barrel 12, and a first plunger 14.

The first needle 11 can move the liquid stored in the second syringe 40 to the first syringe 10. The first needle 11 may be attachable to and detachable from the first syringe barrel 12. The first needle 11 has a tubular shape. The first needle may be formed such that a portion thereof forms an acute angle or an obtuse angle. As a material of the first needle 11, for example, a metal material such as iron or stainless steel can be used. For example, a connection portion of the first needle 11 to the first syringe barrel 12 may be formed of polyethylene, polypropylene, polytetrafluoroethylene, polycarbonate, or the like.

The first syringe barrel 12 stores therein the liquid moved through the first needle 11. In addition, the first syringe barrel 12 can function as a handle portion when an operator operates the first syringe 10. The first syringe barrel 12 has a tubular region. The first syringe barrel 12 can store the liquid in an internal space of the tubular region. The first syringe barrel 12 may have a protruding region protruding from one surface of the tubular region. An internal space of the protruding region is connected to the internal space of the tubular region. The internal space of the protruding region communicates with an internal space of the first needle 11. The protruding region of the first syringe barrel 12 and the first needle 11 may be attachable to and detachable from each other. As a material of each configuration of the first syringe barrel 12, for example, polyethylene, polypropylene, polymethyl methacrylate, polyethylene terephthalate, polycarbonate, or the like can be used.

The first gasket 13 is positioned inside the first syringe barrel 12 and reciprocates in the first syringe barrel 12. The reciprocating movement of the first gasket 13 may be performed via the first plunger 14 by an operator, for example. The first gasket 13 can introduce the liquid into the first syringe barrel 12 and discharge the liquid to the outside of the first syringe barrel 12 through the first needle 11 by reciprocating within the first syringe barrel 12. The first gasket 13 may have a recessed region to which the first plunger 14 can be attached. As a material of the first gasket 13, for example, natural rubber, styrene-butadiene rubber, acrylonitrile rubber, butyl rubber, chloroprene-based rubber, ethylene-propylene rubber, isoprene rubber, urethane rubber, silicone rubber, fluoro rubber, or the like can be used.

The first plunger 14 is a rod-shaped member. An operator can introduce the liquid into the first syringe barrel 12 and discharge the liquid to the outside of the first syringe barrel 12 by operating the first plunger 14. By operating the first plunger 14, the first gasket 13 is reciprocated, and the liquid can be introduced into the first syringe barrel 12 and discharged to the outside of the first syringe barrel 12 through the first needle 11.

The first plunger 14 is attached to the first gasket 13. The first plunger 14 and the first gasket 13 are attachable to and detachable from each other. When the first plunger 14 and the first gasket 13 are attachable to and detachable from each other, a tip of the first plunger 14 may have, for example, a structure engageable with a hole of the first gasket 13, a screw structure, or the like. When the tip of the first plunger 14 has a screw structure, the hole of the first gasket 13 has a screw hole structure. As a material of the first plunger 14, for example, polycarbonate, polypropylene, polymethyl methacrylate, polyethylene terephthalate, or the like can be used.

The syringe system 100 may include a first seal 15 that is attachable to a port portion of the first syringe barrel 12 after the first needle 11 is removed from the first syringe 10. For example, the first seal 15 is attached to the protruding region of the first syringe barrel 12 when the first syringe barrel 12 is used as a centrifugation container. The first syringe 10 can function as a centrifugation container by attaching the first seal 15 to the port portion of the first syringe barrel 12. By using the first seal 15, an operator can perform centrifugation processing on a liquid in a state in which the liquid is accommodated in the second syringe barrel 12. As a material of the first seal 15, for example, natural rubber, styrene-butadiene rubber, acrylonitrile rubber, butyl rubber, chloroprene-based rubber, ethylene-propylene rubber, isoprene rubber, urethane rubber, silicone rubber, fluoro rubber, or the like can be used.

The first needle guide 20 can accommodate the first needle 11 therein. The first needle guide 20 may function as a guide for moving the first needle 11 during operation of the first syringe 10. The first needle guide 20 is positioned at a position shifted from the first syringe 10 by a predetermined distance in the longitudinal direction. The first needle guide 20 is positioned on the first needle 11 side with respect to the first syringe barrel 12 in the longitudinal direction.

The first needle guide 20 has a first hole 21 penetrating in the longitudinal direction of the first needle guide 20. The first needle guide 20 includes a first plate 22 positioned at an end portion on the first syringe 10 side and extending from an outer surface thereof. The first needle guide 20 includes a second plate 23 positioned at a position at a predetermined distance in the longitudinal direction from the first plate 22 and extending from an outer surface thereof. A cross section of the first needle guide 20 perpendicular to the longitudinal direction may be circular, rectangular, or cross-shaped. In the present embodiment, a cross section of the first needle guide 20 perpendicular to the longitudinal direction is circular. As a material of the first needle guide 20, for example, polycarbonate, polypropylene, polymethyl methacrylate, polyethylene terephthalate, or the like can be used.

The first hole 21 can accommodate the first needle 11 therein. The first hole 21 accommodates the first needle 11 therein. A length of the first hole 21 in the longitudinal direction is longer than the length of the first needle 11 in the longitudinal direction. A diameter of the first hole 21 is larger than a diameter of the first needle 11. The diameter of the first hole 21 is smaller than a diameter of the first syringe barrel 12. The first hole 21 penetrates through the first needle guide 20. The first hole 21 may be positioned at a central portion in a cross section of the first needle guide 20 perpendicular to the longitudinal direction. The first hole 21 has a first open end 24 positioned on the first syringe 10 side and a second open end 25 positioned on an opposite side to the first open end 24. Sizes of the first open end 24 and the second open end 25 may be the same or different. The first hole 21 has a tubular shape. In the present embodiment, the first hole 21 has a cylindrical shape. The first open end 24 and the second open end 25 are circular.

The first plate 22 and the second plate 23 can function as handle portions when an operator operates the first needle guide 20. In particular, the first plate 22 may function as a handle portion when an operator simultaneously operates the first syringe and the first needle guide 20. The first plate 22 and the second plate 23 may have a plate shape. Further, the second plate 23 is not necessarily required.

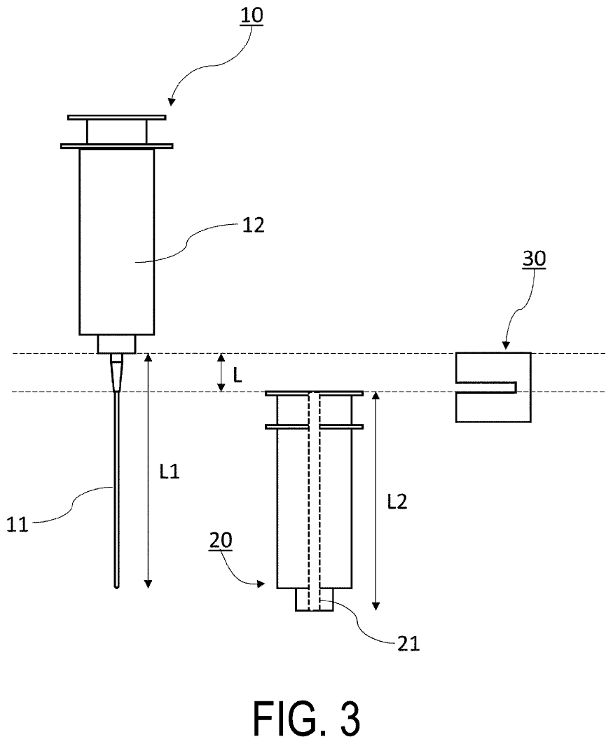

The first holder 30 fixes an interval between the first syringe 10 and the first needle guide 20 at the specific interval L. FIG. 3 illustrates the first holder 30, a positional relationship between the first syringe 10 and the first needle guide 20 in the longitudinal direction, and the lengths of the respective portions. FIG. 3 is a view illustrating a state in which the first syringe 10 and the first needle guide 20 are moved perpendicularly to the longitudinal direction from the state illustrated in FIGS. 1 and 2. The specific interval L is equal to or greater than a difference (L1−L2) between the length L1 of the first needle 11 and the length L2 of the first hole 21 and is equal to or less than the length L1 of the first needle 11. The specific interval L is a length satisfying a relationship “L1≥L≥(L1−L2)”. Here, the interval between the first syringe 10 and the first needle guide 20 refers to a distance between (i) an end portion of the first syringe barrel 12 on the first needle guide 20 side and (ii) an end portion of the first needle guide 20 on the first syringe 10 side in the longitudinal direction of the first syringe 10 and the first needle guide 20. In the present embodiment, the first holder 30 fixes the first needle 11. Since the first holder 30 fixes the first needle 11, it is possible to reduce a concern that the first needle 11 is exposed from the second open end 25 of the first hole 21. The first holder 30 may function as a handle portion when an operator operates the first syringe 10 and the first needle guide 20.

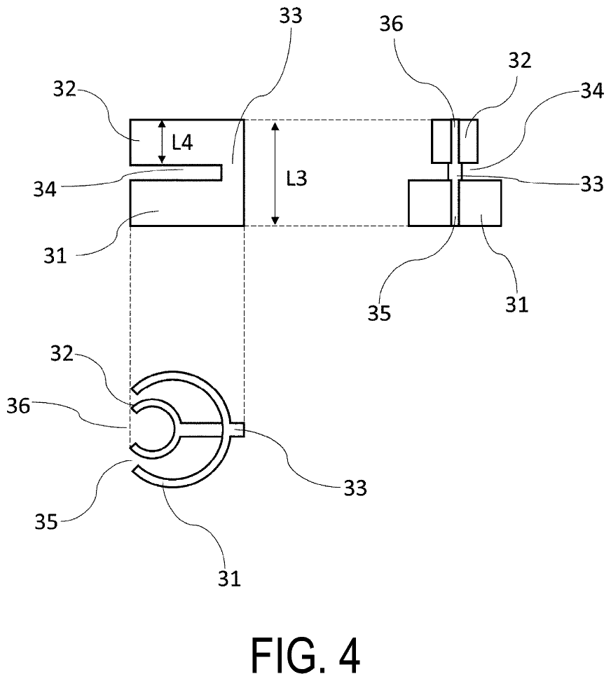

As illustrated in FIG. 4, the first holder 30 includes a first portion 31, a second portion 32 positioned on the first needle guide 20 side with respect to the first portion 31, and a base portion 33 positioned between the first portion 31 and the second portion 32. A length L3 between an end portion of the first portion 31 on the first needle guide 20 side and an end portion of the second portion 32 on the first syringe 10 side in the longitudinal direction is longer than the specific interval L. With this configuration, the first holder 30 can fix the interval between the first syringe 10 and the first needle guide 20 at the specific interval L. As illustrated in FIG. 4, the first holder 30 includes a recessed portion 34 formed by the first portion 31, the second portion 32, and the base portion 33. As a material of the first holder 30, for example, polycarbonate, polypropylene, polymethyl methacrylate, polyethylene terephthalate, or the like can be used.

The first portion 31 fixes the first needle guide 20. The first portion 31 is positioned between the first plate 22 and the second plate 23 of the first needle guide 20. The first portion 31 has a circular arc shape in a cross section perpendicular to the longitudinal direction. With the first portion 31 having this configuration, an operator can easily release the fixing by the first holder 30. The shape of the first portions 431A and 431B in the cross section perpendicular to the longitudinal direction is not limited to the circular arc shape, and may be an L-shape or a U-shape. A central angle of the circular arc of the first portion 31 in the cross section perpendicular to the longitudinal direction is 180° or greater and 360° or less. An inner diameter of the first portion 31 is equal to or less than an outer diameter of the first needle guide 20. The first portion 31, by having this configuration, can firmly fix the first needle guide 20.

The first portion 31 has a first opening portion 35 consisting of two end portions in the shape of a circular arc. A width of the first opening portion 35 is equal to or less than the inner diameter of the first portion 31. With the first opening portion 35 having this configuration, it is possible to reduce a risk that the fixing of the first needle guide 20 by the first holder 30 is released. An inner surface of the first portion 31 is in contact with the first needle guide 20. The inner surface of the first portion 31 may have a shape conforming to an outer surface of the first needle guide 20.

The second portion 32 fixes the first syringe 10. The second portion 32 fixes the first needle 11 of the first syringe 10. The second portion 32 is positioned between the first syringe barrel 12 and the first needle guide 20. A length L4 of the second portion 32 in the longitudinal direction is equal to or greater than the specific interval L.

The second portion 32 has a circular arc shape in a cross section perpendicular to the longitudinal direction. With the second portion 32 having this configuration, an operator can easily release the fixing by the first holder 30. A central angle of the circular arc of the second portion 32 in the cross section perpendicular to the longitudinal direction is 180° or greater and 360° or less. An inner diameter of the second portion 32 is equal to or less than an outer diameter of the first syringe 10. The second portion 32, by having this configuration, allows an inner side of the second portion 32 to press the first syringe 10, thereby firmly fixing the first syringe 10. The inner diameter of the second portion 32 is equal to or less than an outer diameter of the first syringe barrel 12 and the outer diameter of the first needle guide 20. The second portion 32, by having this configuration, can suppress such movement that the first syringe 10 and the first needle guide 20 come close to each other.

The second portion 32 has a second opening portion 36 consisting of two end portions in the shape of a circular arc. A width of the second opening portion 36 is equal to or less than the inner diameter of the second portion 32. With the second opening portion 36 having this configuration, it is possible to reduce the risk that the fixing of the first syringe 10 by the first holder 30 is released. An inner surface of the second portion 32 is in contact with the first syringe 10. The inner surface of the second portion 32 may have a shape conforming to an outer surface of the first syringe 10.

The base portion 33 connects the first portion 31 and the second portion 32. The base portion 33 may function as a handle portion when an operator operates the first holder 30. The base portion 33 may be formed integrally with the first portion 31 and the second portion 32. The base portion 33 has a plate shape. The base portion 33 may have a rod shape.

The recessed portion 34 includes a space positioned between the first portion 31 and the second portion 32. The first plate 22 can be positioned inside the recessed portion 34. By positioning the first plate 22 inside the recessed portion 34, the first portion 31 and the second portion 32 can suppress movement of the first needle guide 20 when the first needle guide 20 attempts to move in the longitudinal direction. The syringe system 100, by having the recessed portion 34, can improve safety when an operator operates the syringe system 100.

Here, in a case where the syringe system does not include the first holder, the first syringe and the first needle guide are provided in an uncombined state. The first needle of the first syringe is provided with a cap mounted thereto. In order to combine the first syringe and the first needle guide, an operator needs to remove the cap on the first needle and then combine the first syringe and the first needle guide by himself/herself. For this reason, there is a problem in that safety when the operator operates the syringe system is low. In particular, when the operator removes the cap on the first needle and combines the first syringe and the first needle guide, there is a concern that the first needle becomes accidentally stuck in a hand, a finger, or the like. On the other hand, even if the first syringe and the first needle guide are provided in a combined state in order to address the above problem, there is a concern that the first needle becomes exposed from the second open end of the first needle guide.

In contrast, as described above, the syringe system 100 includes the first holder 30 that fixes the first syringe 10 and the first needle guide 20 at the specific interval L. The specific interval L is equal to or greater than the difference (L1−L2) between the length L1 of the first needle 11 and the length L2 of the first hole 21. Accordingly, it is possible to reduce the concern that the first needle 11 longer than the first hole 21 is exposed from the second open end 25 of the first hole 21, and to improve safety when the operator operates the syringe system 100.

Furthermore, in the syringe system 100, the specific interval L is equal to or less than the length L1 of the first needle 11. Accordingly, it is possible to reduce the concern that the first needle is exposed from the first open end 24 of the first hole 21, and to improve safety when the operator operates the syringe system 100.

As illustrated in FIGS. 5 and 6, the syringe system 100 may further include the second syringe 40. The second syringe 40 can store a liquid sample. The second syringe 40 can fractionate a plurality of components having different specific gravities in the liquid sample by centrifugation or the like. The first syringe includes a second syringe barrel 42. The second syringe 40 may include a second needle 41 attachable to and detachable from the second syringe barrel 42, a second gasket 43 positionable in the second syringe barrel 42, a second plunger 44, and a second seal 45.

The second syringe barrel 42 stores therein the liquid that has moved through the second needle 41. The first needle guide 20 is positionable inside the second syringe barrel 42. The second syringe barrel 42 may have the same structure as that of the first syringe barrel 12. The second needle 41 may have the same structure and function as the first needle 11. The second gasket 43 may have the same structure and function as the first gasket 13. The second plunger 44 may have the same structure and function as the first plunger 14. The second seal 45 may have the same structure and function as the first seal 15.

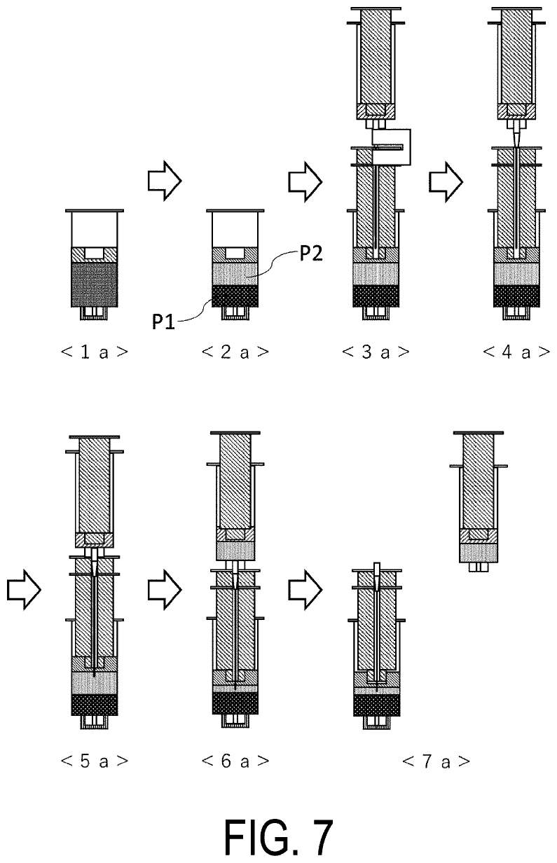

Hereinafter, as an example of a fractionation operation of a liquid sample using the syringe system 100, processing of preparing PRP will be described with reference to FIG. 7. FIG. 7 is a view illustrating an example of a flow of processing of preparing PRP from blood by using the syringe system 100 illustrated in FIGS. 5 and 6.

### Blood Collection Step <1a>

An operator collects blood of a patient using the second syringe 40. The operator removes the second needle 41 from the second syringe barrel 42 and attaches the second seal 45. The operator removes the second plunger 44 from the second gasket 43. <1a> shows a state where the collected blood is stored in the second syringe 40 and the second seal 45 is attached to the second syringe barrel 42.

### First Centrifugation Processing Step <2a>

The operator uses the second syringe 40 as a centrifugation container to perform centrifugation processing using a centrifugal force directed from the second gasket 43 side toward the second seal 45 side. The conditions for the centrifugation processing can be set by the operator in accordance with the use environment of the syringe system 100, and may be, for example, a centrifugal acceleration of 600×g and a processing time of 7 minutes. The liquid in the second syringe barrel 42 is fractionated into a heavy layer H3 containing red blood cells and a light layer L3 containing more platelets than the layer P1. <2a> shows a state of the second syringe 40 and the liquid in the second syringe 40 after the centrifugation processing.

### First Assembly Step <3a>

The operator inserts the first needle guide 20 into the second syringe barrel 42 in a state in which the first syringe 10 and the first needle guide 20 are fixed by the first holder 30. The operator attaches the first needle guide 20 to the second gasket 43. <3a> shows a state of the syringe system 100 in which the first needle guide 20 is attached to the second gasket 43.

### First Releasing Step <4a>

The operator removes the first holder 30 from the first syringe 10 and the first needle guide 20. By removing the first holder 30 from the first syringe 10 and the first needle guide 20, the first syringe 10 becomes movable in the longitudinal direction with respect to the first needle guide 20. <4a> shows a state of the syringe system 100 in which the first holder 30 is removed from the first syringe 10 and the first needle guide 20.

### First Insertion Step <5a>

The operator moves the first syringe 10 so that the distance between the first syringe 10 and the first needle guide 20 in the longitudinal direction is shortened. In the syringe system 100, the length L1 of the first needle 11 is set such that when the first syringe 10 and the first needle guide 20 are brought into contact with each other, an end of the first needle 11 penetrates through a bottom portion of the second gasket 43 and is exposed to the layer P2. <5a> shows a state of the syringe system 100 in which the first holder 30 is removed and the first syringe 10 and the first needle guide 20 are in contact with each other.

### First Extraction Step <6a>

The operator moves the first plunger 14 in a direction in which the first plunger 14 is pulled out from the first syringe barrel 12. Alternatively, the operator pushes down the first syringe barrel 12 toward the second syringe side with respect to the first plunger 14. As a result, the first gasket 13 is moved, and the layer P2 is accommodated in the first syringe 10. By this operation, the operator can separate the layer P2 fractionated from the blood into the first syringe 10. <6a> shows a state in which the layer P2 is separated into the first syringe 10.

### First Separation Step <7a>

The operator removes the first needle 11 from the first syringe barrel 12 in <6a>. Thus, the operator can use the first syringe 10 accommodating the layer P2 for subsequent operations without pulling out the first syringe 10 from the first needle guide 20. For example, by attaching the first seal 15 to the tip of the first syringe 10, the operator can perform centrifugation processing using a centrifugal force directed from the first gasket 13 side toward the first seal 15 side, in which the first syringe 10 is used as a centrifugation container, for further fractionation operation for the layer P2. <7a> shows a state in which the first needle 11 is removed from the first syringe barrel 12.

The fractionation operation is a method of using a syringe system, including inserting the first needle guide 20 into the second syringe barrel 42 in a state in which the first holder 30 fixes the first syringe 10 and the first needle guide 20 at the specific interval L, releasing the fixing by the first holder 30 from the first syringe 10 and the first needle guide 20 in a state in which the first needle guide 20 is positioned inside the second syringe barrel 42, and setting a distance between the first syringe 10 and the first needle guide 20 to be shorter than the specific interval L in a state in which the fixing by the first holder 30 is released from the first syringe 10 and the first needle guide 20. In addition, in the syringe system 100, the specific interval L is equal to or greater than the difference between the length L1 of the first needle 11 of the first syringe 10 and the length L2 of the first hole 21 of the first needle guide 20, and the length L1 of the first needle 11 in the longitudinal direction is longer than the length L2 of the first hole 21 in the longitudinal direction.

In the method of using a syringe system, an example of the step of inserting the first needle guide 20 into the second syringe barrel 42 in a state where the first holder 30 fixes the first syringe 10 and the first needle guide 20 at the specific interval L is the above-described first assembly step. An example of the step of releasing the fixing by the first holder 30 from the first syringe 10 and the first needle guide 20 in a state where the first needle guide 20 is positioned inside the second syringe barrel 42 is the above-described first release step. An example of the step of setting the distance between the first syringe 10 and the first needle guide 20 to be shorter than the specific interval L in a state where the fixing by the first holder 30 is released from the first syringe 10 and the first needle guide 20 is the above-described first insertion step.

By using the above-described method of using a syringe system, an operator can operate the syringe system 100 without exposing the first needle 11 to the outside, whereby safety when the operator uses the syringe system 100 can be improved. For example, the operator can prepare PRP from blood without exposing the first needle 11 to the outside by using the above-described method of using a syringe system.

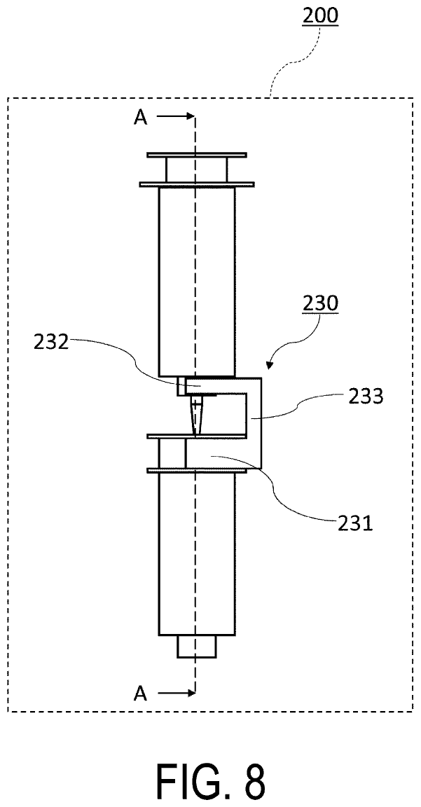

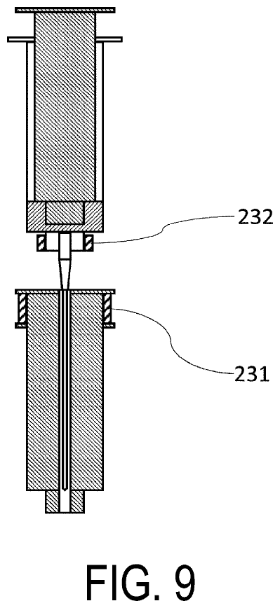

Below, a syringe system 200 according to a second embodiment will be described with reference to FIGS. 8 and 9. FIG. 8 is an overall view of the syringe system 200. FIG. 9 illustrates an internal structure of the syringe system 200.

For convenience of description, members having the same functions as the members described in the above-described embodiment are denoted by the same reference signs, and description thereof is not repeated. For a configuration corresponding to (similar to) the configuration of the embodiment described above, even when a reference sign different from that of the configuration of the embodiment described above is denoted, matters not particularly mentioned are the same as or similar to the configuration of the embodiment described above.

The syringe system 200 is different from the syringe system 100 in terms of the structure and function of a first holder 230. The first holder 230 fixes the first syringe barrel 12 of the first syringe 10 and the first needle guide 20. The first holder 230, by having this configuration, can stabilize the first syringe 10 when an operator operates the syringe system 200.

The first holder 230 can shorten the distance between the first syringe 10 and the first needle guide 20 from the specific interval L in a state where the first syringe 10 and the first needle guide 20 are fixed. In addition, when the distance between the first syringe 10 and the first needle guide 20 is shorter than the specific interval L, the first holder 230 can apply a force to the first syringe 10 and the first needle guide 20 in a direction in which the distance therebetween increases.

A second portion 232 fixes the first syringe barrel 12. The second portion 232 fixes a protruding region protruding from one surface of the tubular region of the first syringe barrel 12. In a case where the second portion 232 fixes the protruding region of the first syringe barrel 12, when the first syringe 10 and the first needle guide 20 move in an approaching direction, the second portion 232 and the bottom portion of the first syringe barrel 12 come into contact with each other. Therefore, the first holder 230, by having this configuration, can reduce the risk that the first needle 11 is exposed. The second portion 232 may fix the tubular portion of the first syringe barrel 12. In a case where the second portion 232 fixes the tubular portion of the first syringe barrel 12, it is possible to further reduce shaking of the first syringe 10 when an operator operates the syringe system 200.

A base portion 233 is stretchable in the longitudinal direction. The base portion 233 can elastically apply a force to a first portion 231 and the second portion 232 in the longitudinal direction. The syringe system 200, by having this configuration, can be used for the fractionation operation without releasing the fixing by the first holder 230 while reducing the risk that the first needle 11 is unintentionally exposed by an operator. Therefore, it is possible to reduce the labor of the operator, and to reduce the risk of injury caused by the first needle 11 when the operator releases the fixing by the first holder 230. In particular, in a case where the distance between the first syringe 10 and the first needle guide 20 is shorter than the specific interval L, the base portion 233 may apply a force to the first portion 231 and the second portion 232 in the longitudinal direction so that the distance therebetween increases. When the distance between the first syringe 10 and the first needle guide 20 is shorter than the specific interval L, the base portion 233 may apply a force to the first portion 231 and the second portion 232 in a direction in which the distance between the first syringe 10 and the first needle guide 20 approaches the specific interval L. The base portion 233 may be configured by a coil spring, a plate spring, or the like.

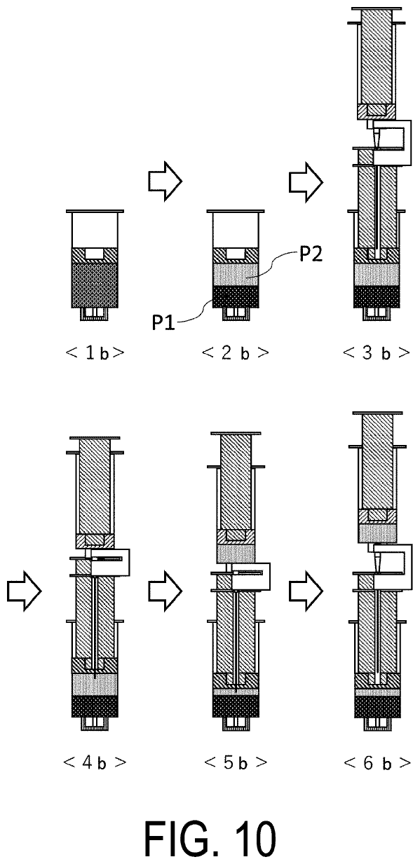

Hereinafter, as an example of a fractionation operation of a liquid sample using the syringe system 200, processing of preparing PRP will be described with reference to FIG. 10. FIG. 10 is a view illustrating an example of a flow of preparing PRP from blood by using the syringe system 200 illustrated in FIGS. 8 and 9. The blood collection step <1b> to the first assembly step <3b> are the same as or similar to the steps <1a> to <3a> in the syringe system 100, and therefore description thereof will be omitted. The first extraction step <5b> is the same as or similar to the first extraction step <6a> in the syringe system 100 except that the first holder 230 is in a mounted state, and thus description thereof will be omitted.

### First Insertion Step <4b>

The operator moves the first syringe 10 so that the distance between the first syringe 10 and the first needle guide 20 in the longitudinal direction decreases, while maintaining a state in which the first holder 230 fixes the first syringe 10 and the first needle guide 20. <4b> shows a state of the syringe system 200 in which the first syringe 10 and the first needle guide 20 are in contact with the first holder 230.

### First Separation Step <6b>

The operator returns the distance between the first syringe 10 and the first needle guide 20 to the specific interval L while maintaining the state in which the first holder 230 fixes the first syringe 10 and the first needle guide 20. <6b> shows a state after the distance between the first syringe 10 and the first needle guide 20 is returned to the specific interval L.

After the first separation step <6b>, the operator removes the first holder 230 from the first syringe 10 and the first needle guide 20, and can use the first syringe 10 in which the layer P2 is accommodated for subsequent operations. For example, the operator can administer the layer P2 to a subject using the first syringe 10. In addition, the operator may replace the first needle 11 with another needle before administering the layer P2. When discarding the layer P2, the operator can discard the first syringe without removing the first needle guide 20 from the first syringe 10 after the first separation step <6b>. According to this operation, the first syringe can be discarded without exposing the first needle 11 of the first syringe 10, which can improve safety when the operator uses the syringe system 200.

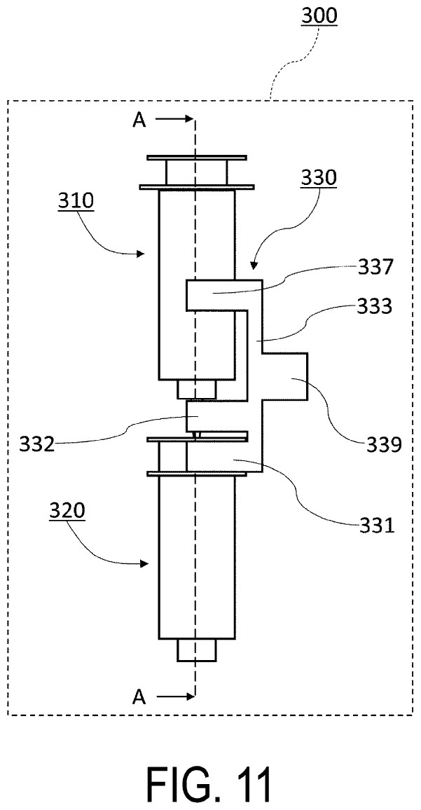

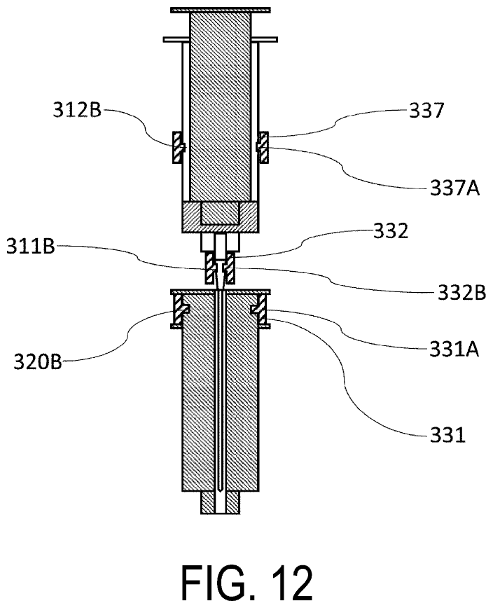

Below, a syringe system 300 according to a third embodiment will be described with reference to FIGS. 11 and 12. FIG. 11 is an overall view of the syringe system 300. FIG. 12 illustrates an internal structure of the syringe system 300.

For convenience of description, members having the same functions as those of the members described in the above-described embodiment are denoted by the same reference signs, and description thereof is not repeated. For a configuration corresponding to (similar to) the configuration of the embodiment described above, even when a reference sign different from that of the configuration of the embodiment described above is denoted, matters not particularly mentioned are the same as or similar to the configuration of the embodiment described above.

The syringe system 300 is different from the syringe system 100 in terms of the structure and function of a first holder 330. The first holder 330 fixes the first syringe barrel 12 of the first syringe 10, the first needle 11, and a first needle guide 320. The first holder 230, by having this configuration, can reduce shaking of the first syringe 10 when an operator operates the syringe system 300, and strengthen the fixing by the first holder 330.

A second portion 332 of the first holder 330 fixes the first needle 11. The first holder 330 includes a third portion 337 for fixing the first syringe barrel 12. The third portion 337 has a circular arc shape in a cross section perpendicular to the longitudinal direction. A central angle of the circular arc of the third portion 337 in the cross section perpendicular to the longitudinal direction is 180° or greater and 360° or less. An inner diameter of the third portion 337 is equal to or less than the outer diameter of the first syringe barrel 12.

The third portion 337 has a third opening portion 338 consisting of two end portions in the shape of a circular arc. A width of the third opening portion 338 is equal to or less than the inner diameter of the third portion 337. An inner surface of the third portion 337 is in contact with the first syringe barrel 12. The inner surface of the third portion 337 may have a shape conforming to the outer surface of the first syringe barrel 12.

As illustrated in FIG. 11, the first holder 330 includes a grip portion 339 that can be gripped by an operator. The grip portion 339 extends from a base portion 333 of the first holder 330. The grip portion 339 may extend from a portion other than the base portion 333 of the first holder 330. The grip portion 339 extends toward a side opposite to the first syringe 10 with respect to the base portion 333. The grip portion 339 may be attachable to and detachable from the base portion 333. The first holder 330 may have a plurality of grip portions 339. With the syringe system 300 having this configuration, the operator can grip the grip portion 339 and release the fixing by the first holder 30. The syringe system 300, by having this configuration, improves operability when an operator uses the syringe system 300.

As illustrated in FIG. 12, an inner surface of the first portion 331 is a contact surface with the first needle guide 320. In addition, the inner surface of the first portion 331 has a first protruding portion 331A that comes into contact with the outer surface of the first needle guide 320. The first protruding portion 331A may be positioned only on a part of the inner surface of the first portion 331. The first protruding portion 331A may be positioned along an inner peripheral surface of the first portion 331. The first protruding portion 331A may be positioned parallel to an end portion of the first portion 331 on the first syringe 310 side. The first needle guide 320 has a first recessed portion 320B engageable with the first protruding portion 331A. The syringe system 300, by having this configuration, can strengthen the fixing between the first holder 330 and the first needle guide 320. The first needle guide 20 may have the first protruding portion 331A, and the first portion 331 may have the first recessed portion 320B.

The inner surface of the second portion 332 or the inner surface of the third portion 337 has a second protruding portion 332B or a third protruding portion 337A in the same manner as or similar to the first portion 331. A first needle 311 or a first syringe barrel 312 may have a second recessed portion 311B or a third recessed portion 312B in the same manner as or similar to the first needle guide 320. The syringe system 300, by having this configuration, can strengthen the fixing between the first holder 330 and the first syringe 310.

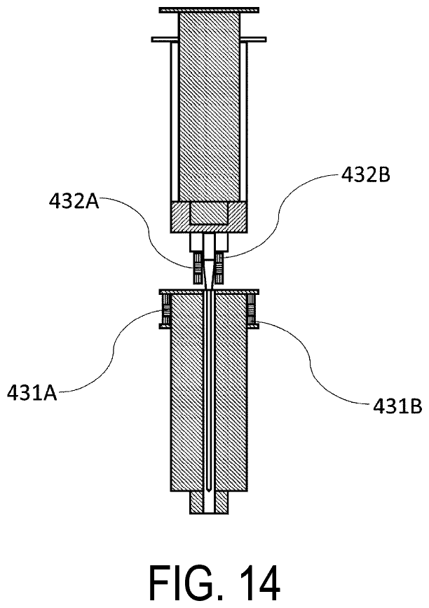

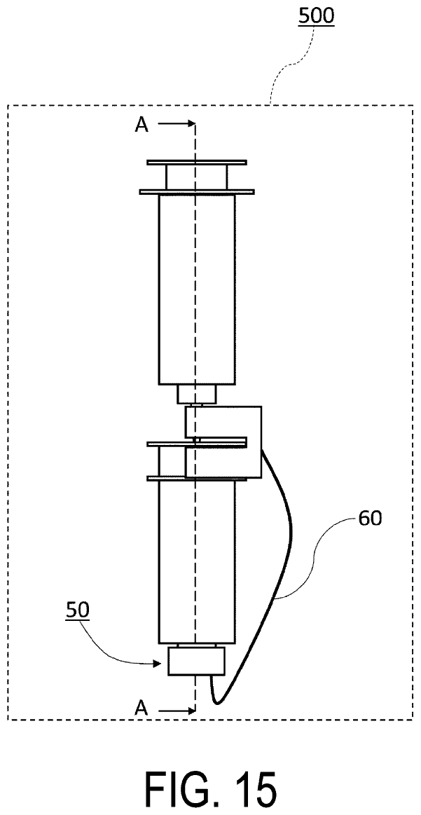

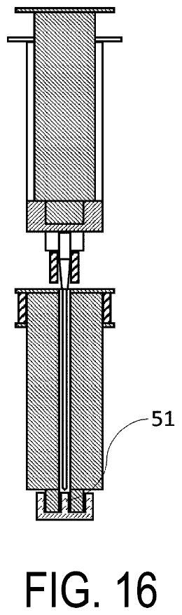

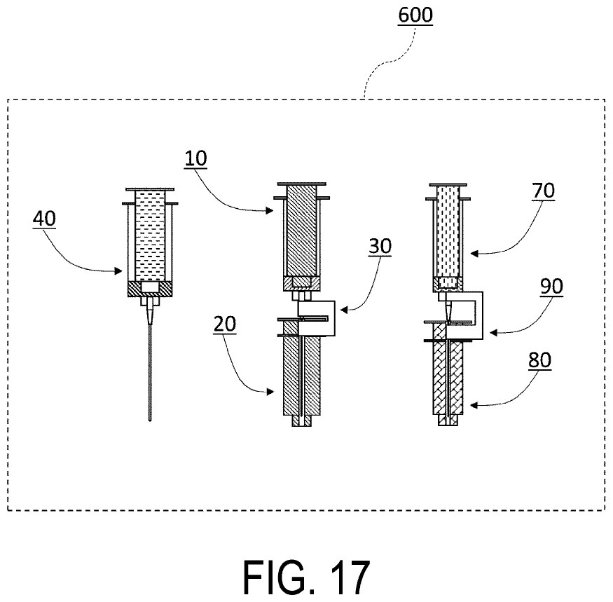

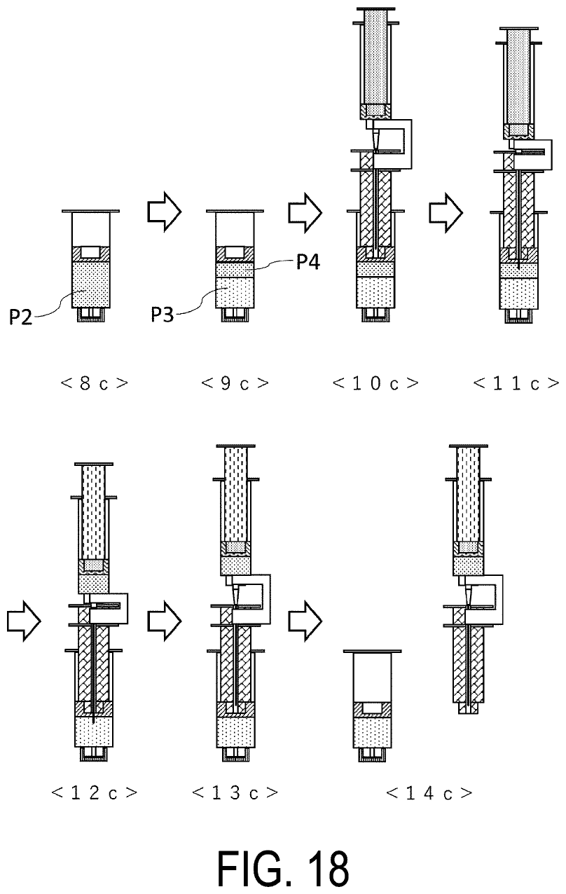

Below, a syringe system 400 according to a fourth embodiment will be described with reference to FIGS. 13 and 14. FIG. 13 is an overall view of the syringe system 400. FIG. 14 illustrates an internal structure of the syringe system 400.

For convenience of description, members having the same functions as those of the members described in the above-described embodiment are denoted by the same reference signs, and description thereof is not repeated. For a configuration corresponding to (similar to) the configuration of the embodiment described above, even when a reference sign different from that of the configuration of the embodiment described above is denoted, matters not particularly mentioned are the same as or similar to the configuration of the embodiment described above.

The syringe system 400 is different from the syringe system 100 in terms of the structure and function of a first holder 430. The syringe system 400 includes a first member 430A and a second member 430B, as the first holder 430. The first member 430A and the second member 430B have first portions 431A and 431B, second portions 432A and 432B, and base portions 433A and 433B, respectively. The first portions 431A and 431B can be fitted with each other. The second portions 432A and 432B can be fitted with each other. The syringe system 300, by having this configuration, can strengthen the fixing by the first holder 430. With the syringe system 400 having this configuration, an operator can easily perform an operation for releasing the fixing by the first holder 430.

The first portions 431A and 431B can be fitted with each other at end portions thereof. The first portion 431A has a protruding shape at an end portion. The first portion 431B has a recessed shape at an end portion. The protruding shape of the first portion 431A and the recessed shape of the first portion 431B can fit with each other. The end portions of the first portions 431A and 431B are not limited to the protruding shape and the recessed shape, and may have, for example, a structure in which a claw and a hole are engaged with each other, or a structure in which holes are respectively formed and fixed by a screw or the like. A central angle of a circular arc of each of the first portions 431A and 431B in the cross section perpendicular to the longitudinal direction is 180°. The central angle of the circular arc of the first portions 431A and 431B in the cross section perpendicular to the longitudinal direction is not limited to 180°. For example, the central angle of the first portion 431A may be 270°, and the central angle of the first portion 431B may be 90°.

The second portions 432A and 432B have the same structures and functions as the first portions 431A and 431B, respectively. The structures of the second portions 432A and 432B may be different from the structures of the first portions 431A and 431B. For example, only the second portions 432A and 432B may be fittable with each other. The second portions 432A and 432B may not be fitted to each other, and only the first portions 431A and 431B may be fittable with each other.

Below, a syringe system 500 according to a fifth embodiment will be described with reference to FIGS. 15 and 16. FIG. 15 is an overall view of the syringe system 500. FIG. 16 illustrates an internal structure of the syringe system 500.

For convenience of description, members having the same functions as those of the members described in the above-described embodiment are denoted by the same reference signs, and description thereof is not repeated. For a configuration corresponding to (similar to) the configuration of the embodiment described above, even when a reference sign different from that of the configuration of the embodiment described above is denoted, matters not particularly mentioned are the same as or similar to the configuration of the embodiment described above.

The syringe system 500 is different from the syringe system 100 in that it includes a cap 50 and a coupling member 60. The cap 50 may cover the second open end 25 of the first needle guide 20. The cap 50 is attachable to and detachable from the first needle guide 20. The syringe system 500, by having the cap 50, reduces the risk that an end portion of the first needle 11 is exposed from the first hole 21, and improves safety when an operator operates the syringe system 500. The syringe system 500, by having the cap 50, can reduce the risk that an end portion of the first needle 11 is exposed from the second open end 25 of the first hole 21, for example, even when the fixing by the first holder 30 is unintentionally released.

The cap 50 has a protrusion 51 that is accommodated in the first hole 21 from the second open end 25. As a material of the cap 50, for example, butyl rubber, urethane rubber, fluoro rubber, silicone rubber, butadiene rubber, ethylene-propylene rubber, acrylic rubber, or the like can be used. By using the above-described material as the material of the cap 50, the cap 50 gains stretchability, reducing the risk that the cap 50 comes off when the first needle 11 attempts to move toward the second open end 25 side. Therefore, by using the above-described material as the material of the cap 50, safety when an operator operates the syringe system 500 is improved.

The protrusion 51 is accommodated in the first hole 21 from the second open end 25 and can fill a part of the first hole 21. When the first needle 11 moves toward the second open end 25 side, the protrusion 51 comes into contact with the first needle 11. When the first needle 11 moves to the second open end 25 side in a state of being in contact with the protrusion 51, the protrusion 51 may be deformable. In addition, when the first needle 11 moves to the second open end 25 side in a state of being in contact with the protrusion 51, the protrusion 51 may be able to apply a force to the first needle 11 toward the first open end 24 side. The cap 50, by having the protrusion 51, can reduce the risk that it is detached from the first needle guide 20 as the first needle 11 moves toward the second open end 25 side. The cap 50, by having the protrusion 51, can reduce the risk that the first needle 11 penetrates through the cap 50 as the first needle 11 moves toward the second open end 25 side. Therefore, with the cap 50 having the protrusion 51, safety when an operator operates the syringe system 500 is improved.

The coupling member 60 couples the first holder 30 and the cap 50. The coupling member 60 is coupled to the base portion 33 of the first holder 30. The coupling member 60 is coupled to the cap 50. The coupling member 60 may be attachable to and detachable from the first holder 30 or the cap 50. The coupling member 60 may have stretchability. As a material of the coupling member 60, for example, silicone rubber, natural rubber, or the like can be used. The syringe system 500, by having the coupling member 60, can reduce the risk of losing the first holder 30 or the cap 50 when an operator operates the syringe system 500. For example, the operator needs to remove the first holder 30 or the cap 50 from the first syringe 10 or the first needle guide 20 during the fractionation operation of the liquid sample using the syringe system 500.

Below, a syringe system 600 according to a sixth embodiment will be described with reference to FIGS. 17 and 18. FIG. 17 is a configuration example of the syringe system 600. FIG. 18 illustrates an example of a fractionation operation of a liquid sample using the syringe system 600.

For convenience of description, members having the same functions as those of the members described in the above-described embodiment are denoted by the same reference signs, and description thereof is not repeated. For a configuration corresponding to (similar to) the configuration of the embodiment described above, even when a reference sign different from that of the configuration of the embodiment described above is denoted, matters not particularly mentioned are the same as or similar to the configuration of the embodiment described above.

The syringe system 600 is different from the syringe system 100 in that it includes a third syringe 70, a second needle guide 80, and a second holder 90. The third syringe 70 has the same structure and function as the first syringe 10. The second needle guide 80 has the same structure and function as the first needle guide 20. The second holder 90 has the same structure and function as the first holder 230. The second holder 90 may have the same structure and function as the first holder 30, 330, 430.

Hereinafter, as an example of a fractionation operation of a liquid sample using the syringe system 600, processing of preparing PRP will be described with reference to FIG. 18. The blood collection step <1c> to the first separation step <7c> are the same as or similar to the blood collection step <1a> to the first separation step <7a> in the syringe system 100, and therefore description thereof will be omitted.

### First Attachment Step <8c>

The operator attaches the first seal 15 to the first syringe 10 in which the layer P2 is stored. The operator removes the first plunger 14 from the first gasket 13. <8c> shows a state in which the collected layer P2 is stored in the first syringe 10 and the first seal 15 is attached to the first syringe barrel 12.

### Second Centrifugation Step <9c>

The operator uses the first syringe 10 as a centrifugation container to perform centrifugation processing using a centrifugal force directed from the first gasket 13 side toward the first seal 15 side. The conditions for the centrifugation processing can be set by the operator in accordance with the use environment of the syringe system 100, and may be, for example, a centrifugal acceleration of 2000×g and a processing time of 5 minutes. The liquid in the first syringe barrel 12 is fractionated into a layer P3 containing platelets at a higher concentration than in the layer P2 and a layer P4 containing platelets at a lower concentration than in the layer P2. <9c> shows a state of the first syringe 10 and the liquid in the first syringe 10 after the centrifugation processing.

### Second Assembly Step <10c>

The operator inserts the second needle guide 80 into the first syringe barrel 12 in a state in which the third syringe 70 and the second needle guide 80 are fixed by the second holder 90. The operator attaches the second needle guide 80 to the first gasket 13. <10c> shows a state of the syringe system 600 in which the second needle guide 80 is attached to the first gasket 13.

### Second Insertion Step <11c> to Second Separation Step <13c>

The second insertion step <11c> to the second separation step <13c> are the same as or similar to the first insertion step <4b> to the first separation step <6b> in the syringe system 200, and therefore description thereof will be omitted. The first syringe 10 in the syringe system 600 corresponds to a syringe that stores blood of a patient and for which centrifugation processing is performed, in a blood fractionation operation using the syringe system 200 (see FIG. 10). The third syringe 70 in the syringe system 600 corresponds to the first syringe 10 in the syringe system 200. The second needle guide 80 in the syringe system 600 corresponds to the first needle guide 20 in the syringe system 200. The second holder 90 in the syringe system 600 corresponds to the first holder 230 in the syringe system 200. The layer P4 in the second insertion step <11c> to the second separation step <13c> corresponds to the layer P2 in the first insertion step <4b> to the first separation step <6b>.

### First Disassembly Step <14c>

The operator removes the second needle guide 80 from the second gasket 43 in a state in which the third syringe 70 and the second needle guide 80 are fixed to the second holder 90. The layer P3 is accommodated in the first syringe 10, and the layer P4 is accommodated in the third syringe 70. <14c> shows a state after the second needle guide 80 is removed from the first gasket 13.

By using the syringe system 600, the operator can perform fractionation operations of more varied liquid samples. For example, in the preparation of PRP using the syringe system 600, it is possible to obtain the layer P3 in which platelets are further concentrated from the layer P2 separated in the blood collection step <1c> to the first separation step <7c>.

The invention according to the present disclosure has been described above based on the drawings and examples. However, the invention according to the present disclosure is not limited to each embodiment described above. That is, the invention according to the present disclosure can be modified in various ways within the scope illustrated in the present disclosure, and embodiments obtained by appropriately combining the technical means disclosed in different embodiments are also included within the technical scope of the invention according to the present disclosure. In other words, those skilled in the art can easily make various variations or modifications based on the present disclosure. Note that these variations or modifications are included within the scope of the present disclosure.

### REFERENCE SIGNS

100, 200, 300, 400, 500, 600 Syringe system
10, 310 First syringe
11, 311 First needle
311B Second recessed portion
12, 312 First syringe barrel
312B Third recessed portion
13 First gasket
14 First plunger
15 First seal
20, 320 First needle guide
320B First recessed portion
21 First hole
22 First plate
23 Second plate
24 First open end
25 Second open end
30, 230, 330, 430 First holder
430A First member
430B Second member
31, 231, 331, 431A, 431B First portion
331A First protruding portion
32, 232, 332, 432A, 432B Second portion
332B Second protruding portion
33, 233, 333, 433A, 433B Base portion
34 Recessed portion
35 First opening portion
36 Second opening portion
337 Third portion
337A Third protruding portion
338 Third opening portion
339 Grip portion
40 Second syringe
41 Second needle
42 Second syringe barrel
43 Second gasket
44 Second plunger
45 Second seal
50 Cap
51 Protrusion
60 Coupling member
70 Third syringe
80 Second needle guide
90 Second holder

1. A syringe system comprising:
a first syringe comprising a first needle;
a first needle guide comprising a first hole in which the first needle is positioned and which is shorter than a length of the first needle in a longitudinal direction; and
a first holder configured to fix the first syringe and the first needle guide, wherein
a length between the first syringe and the first needle guide is a specific interval, and
the specific interval is greater than a difference between the length of the first needle and a length of the first hole.

2. The syringe system according to claim 1, wherein
the first holder is configured to fix the first needle.

3. The syringe system according to claim 1, wherein
the specific interval is shorter than the length of the first needle.

4. The syringe system according to claim 1, wherein
the first holder comprises a first portion and a second portion,
the first portion is configured to fix the first needle guide,
the second portion is configured to fix the first syringe, and
a length between an end portion of the first needle guide on the first syringe side and an end portion of the second portion on the first syringe side is longer than the specific interval.

5. The syringe system of claim 4, wherein
an inner diameter of the first portion is equal to or less than an outer diameter of the first needle guide, and
an inner diameter of the second portion is equal to or less than an outer diameter of the first syringe.

6. The syringe system according to claim 4, wherein
the first syringe comprises a first syringe barrel connected to the first needle, and
an inner diameter of the second portion is smaller than an outer diameter of the first syringe barrel and an outer diameter of the first needle guide.

7. The syringe system according to claim 4, wherein
the first portion comprises a first opening portion having a width smaller than an inner diameter of the first portion, and
the second portion comprises a second opening portion having a width smaller than an inner diameter of the second portion.

8. The syringe system according to claim 4, wherein
the first needle guide comprises a first plate at an end portion on the first syringe side, and
the first holder comprises a recessed portion positioned between the first portion and the second portion, the first plate being positioned in the recessed portion.

9. The syringe system according to claim 4, wherein
a surface of the first portion that comes into contact with the first needle guide has a shape conforming to an outer surface of the first needle guide.

10. The syringe system according to claim 4, wherein
a surface of the second portion that comes into contact with the first syringe has a shape conforming to an outer surface of the first syringe.

11. The syringe system according to claim 4, wherein
the first holder comprises a base portion positioned between the first portion and the second portion.

12. The syringe system of claim 11, wherein
the base portion is configured to be capable of elastically applying a force to the first portion and the second portion in the longitudinal direction.

13-14. (canceled)

15. The syringe system according to claim 4, wherein
the syringe system comprises two first holders, and
the first portions and/or the second portions of the two first holders are fitted to each other.

16. The syringe system according to claim 4, wherein
the first syringe comprises a first syringe barrel connected to the first needle,
the first holder comprises a third portion configured to fix the first syringe barrel, and
the second portion is configured to fix the first needle.

17. The syringe system according to claim 4, wherein
the first portion comprises a first protruding portion or a first recessed portion on a contact surface with the first needle guide, and
the first needle guide comprises a first protruding portion or a first recessed portion fittable with the first protruding portion or first recessed portion of the first portion.

18. The syringe system according to claim 4, wherein
the second portion comprises a second protruding portion or a second recessed portion on a contact surface with the first syringe, and
the first syringe comprises a second protruding portion or a second recessed portion fittable with the second protruding portion or second recessed portion of the second portion.

19. The syringe system according to claim 1, wherein
the first syringe comprises a first syringe barrel connected to the first needle, and
the first holder is configured to fix the first syringe barrel.

20. (canceled)

21. The syringe system according to claim 1, wherein
the first hole comprises a first open end positioned on the first syringe side and a second open end positioned on a side opposite to the first open end, and
the syringe system further comprises a cap attachable to the first needle guide while covering the second open end.

22.-25 (canceled)

26. The syringe system according to claim 1, further comprising:
a second syringe comprising a second syringe barrel in which the first needle guide is positionable.

27. A method of using a syringe system, comprising:
inserting a first needle guide into a second syringe barrel in a state in which a first holder fixes a first syringe and the first needle guide at a specific interval,
releasing the fixing by the first holder from the first syringe and the first needle guide in a state in which the first needle guide is positioned inside the second syringe barrel, and
setting a distance between the first syringe and the first needle guide to be shorter than the specific interval in a state in which the fixing by the first holder is released from the first syringe and the first needle guide, wherein
the specific interval is greater than a difference between a length of a first hole of the first needle guide and a length of a first needle of the first syringe, and
a length of the first needle in a longitudinal direction is longer than a length of the first hole in the longitudinal direction.
